(19) United States
(12) Patent Application Publication
(10) Pub. No.: US 2023/0158094 A1
(43) Pub. Date: May 25, 2023

(54) Plug Product and a Method of Using the Plug Product for Tooth-Associated Pain

(71) Applicant: Bradford Randall Jones, Bend, OR (US)

(72) Inventor: Bradford Randall Jones, Bend, OR (US)

(21) Appl. No.: 17/531,731

(22) Filed: Nov. 20, 2021

(51) Int. Cl. A61K 36/61; A61K 9/00; A61K 31/33; A61K 47/44; A61K 36/534; A61K 36/185

(52) U.S. Cl. CPC A61K 36/61; A61K 9/0053; A61K 31/33; A61K 47/44; A61K 36/534; A61K 36/185

(57) ABSTRACT

A method of reducing tooth-associated pain includes at least shaping plug product into a shape, identifying a source of tooth-associated pain, and engaging the shaped plug product directly with the source of tooth-associated pain. Some implementations include at least shaping a plug product that is at least partly composed of gum base. Some particular implementations include at least shaping a plug product that is completely composed of gum base. Some other particular implementations include at least shaping a plug product that includes one or more numbing agents. Some alternative implementations, include at least shaping a plug product that is at least partly composed of beeswax. A plug product for reducing tooth-associated pain includes a mixture that includes at least one or more numbing agents, at least one of beeswax or gum base and at least one of stevia, corn starch, glycerin, citric acid, or corn syrup.

### TECHNICAL FIELD

Certain embodiments pertain to a plug product and a method of using a plug product for relief of dental pain, such as nerve pain. In particular, certain embodiments pertain to a plug product and a method of using a plug product that is at least partially composed of at least one of a gum base or bees wax for relief of dental pain, such as nerve pain.

### BACKGROUND

Unfortunately, dental pain is a common human experience. Some dental pain may be referred to as tooth-associated pain, meaning pain that is somehow associated with a tooth. Examples of tooth-associated pain include, without limitation, pain associated with a tooth cavity, a tooth with a missing filling, a tooth that is at least one of cracked or fractured, an at least partly exposed tooth root, an exposed oral nerve, and other causes. Frequently, the tooth-associated pain involves a nerve that is either exposed or deprived or its normal protection by gums or teeth.

While professional dental care is often effective at treating tooth-associated pain, sometimes it is not possible to immediately be seen by a dental professional. And at other times, there is still a need for methods of relieving tooth-associated pain as an adjunct to professional dental care. Thus, it is desirable to provide methods of relieving tooth-associated pain while waiting to be seen by a professional or as an adjunct to professional dental care.

### SUMMARY

This summary is provided as a convenience to the reader as a summary of one or more embodiments. The discussion in this Summary is intentionally simplified. It is not intended as a guide for construing or limiting the scope of the claims.

Some embodiments provide a method of reducing tooth-associated pain. The method includes at least shaping plug product into a shape, identifying a source of tooth-associated pain, and engaging the shaped plug product directly with the source of tooth-associated pain.

Some embodiments include at least shaping a plug product that is at least partly composed of gum base. Some particular embodiments include at least shaping a plug product that is completely composed of gum base. Some other particular embodiments include at least shaping a plug product that includes at least a mixture of at least (1) gum base and (2) at least one of stevia, corn starch, glycerin, citric acid, one or more numbing agents, corn syrup, or beeswax.

Some embodiments include at least shaping a plug product that is at least partly composed of beeswax.

Some embodiments provide a plug product, the plug product including at least a mixture that includes at least (1) one or more numbing agents, (2) at least one of beeswax or gum base, and (3) one or more additional ingredients that in some embodiments include at least one of stevia, corn starch, glycerin, citric acid, or corn syrup.

### BRIEF DESCRIPTION OF THE DRAWINGS

Various embodiments will be described, by way of example, with reference to the accompanying drawings, in which

FIG. 1 is perspective drawing of a block of plug product, consistent with some embodiments.

FIGS. 2A-2D are two-dimensional drawings showing portions of plug products that have been shaped in a variety of shapes.

FIG. 3A is a perspective drawing of a person experiencing tooth-associated pain.

FIG. 3B is a perspective drawing of a mouth of the person of FIG. 3A showing a fracture and hole in a tooth.

FIG. 3C is a sequence drawing showing a sequence with a portion of plug product, the portion of plug product shaped into a sphere-like shape, and the shaped plug product in preparation for being used to fill the hole in the fractured tooth of FIG. 3B.

FIG. 3D is a perspective partially sectional drawing showing the plug product applied to the fractured tooth of FIG. 3B to plug the hole.

FIG. 4A is a perspective partially sectional drawing showing a mouth with an affected tooth having a receding gum line that partially exposes a tooth root.

FIG. 4A-1 is an enlargement of a portion of FIG. 4 showing an enlargement of the affected tooth with the receding gum line that partially exposes a tooth root.

FIG. 4B is a perspective partially sectional drawing showing a shaped plug product before being applied to the affected tooth of FIG. 4A to at least partially cover the exposed tooth root.

FIG. 4C is a perspective partially sectional drawing showing the plug product applied to cover the exposed tooth root of the affected tooth of FIGS. 4A and 4B.

FIG. 5 is a flow diagram illustrating a method according to some embodiments.

FIG. 6 is a flow diagram illustrating the method of FIG. 5 according to some embodiments, with additional optional operations.

FIG. 7 is a flow diagram illustrating the method of FIG. 5 according to some embodiments, with additional optional operations.

FIG. 8 is a flow diagram illustrating the method of FIG. 5 according to some embodiments, with additional optional operations.

FIG. 9 is a flow diagram illustrating the method of FIG. 5 according to some embodiments, with additional optional operations.

FIG. 10 is a simplified block diagram of a plug product showing potential ingredients.

### DETAILED DESCRIPTION

The above-described drawings are not necessarily to scale. And in most cases are simplified to avoid cluttering the drawings with unimportant details. It is to be understood that the disclosed embodiments are merely exemplary. No attempt is made to describe all possible embodiments, which may be embodied in various forms. It is also to be understood that multiple references to “some embodiments” are not necessarily referring to the same embodiments.

Unfortunately, dental pain is a common human experience. Some dental pain may be referred to as tooth-associated pain, meaning pain that is somehow associated with a tooth. Examples of tooth-associated pain include, without limitation, pain associated with a tooth cavity, a tooth with a missing filling, a tooth that is at least one of cracked or fractured, an at least partly exposed tooth root, an exposed oral nerve, and other causes. Frequently, the tooth-associated pain involves a nerve that is either exposed or deprived or its normal protection by gums or teeth.

Often, it is helpful to have a source of tooth-associated pain at least covered plugged to insulate the source of tooth-associated pain from air, liquids, food, and from hot or cold temperatures.

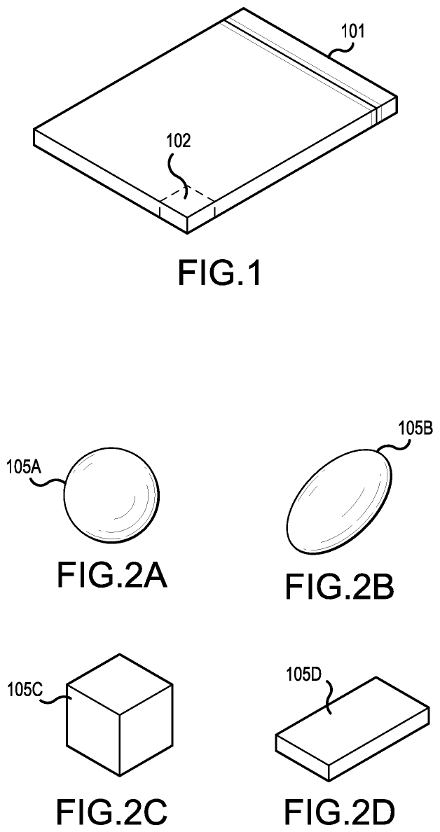

Referencing FIG. 1, a block of plug product 101 is shown in perspective. A “portion” 102 that can be torn or cut off allows for use of a small amount for use on a source of tooth-associated pain. As used in this document, plug product means a product composed of disclosed ingredients which is amenable to being shaped to cover or plug a source of tooth-associated pain.

Referencing FIG. 2A, a torn-off portion 102 (e.g. of FIG. 1) is shown after being shaped into a plug product with a sphere-like shape 105A. Referencing FIG. 2B, a torn-off portion 102 is shown after being shaped into plug product with ellipsoidal-like shape 105B. Referencing FIG. 2C, a torn-off portion 102 is shown after being shaped into plug product with a rectangular shape 105C, for example a rectangular cuboid as shown. Referencing FIG. 21D, a torn-off portion 102 is shown after being shaped into plug product with a flattened shape 105C, for example a another flattened rectangular cuboid as shown. The shapes of FIG. 2A 2D are only examples. Other shapes are possible such as an irregular shape. The word “like” after “shape” is intended to convey that the shape is approximate. For example, a user may roll a portion 102 of plug product into a sphere-like shape with their fingers, but it is very unlikely that the sphere-like shape will be a perfect sphere. in some embodiments, the word “like” refers to being with 40% of a perfect sphere shape, a perfect ellipsoidal shape, a perfect rectangular shape, or other perfectly flat shape.

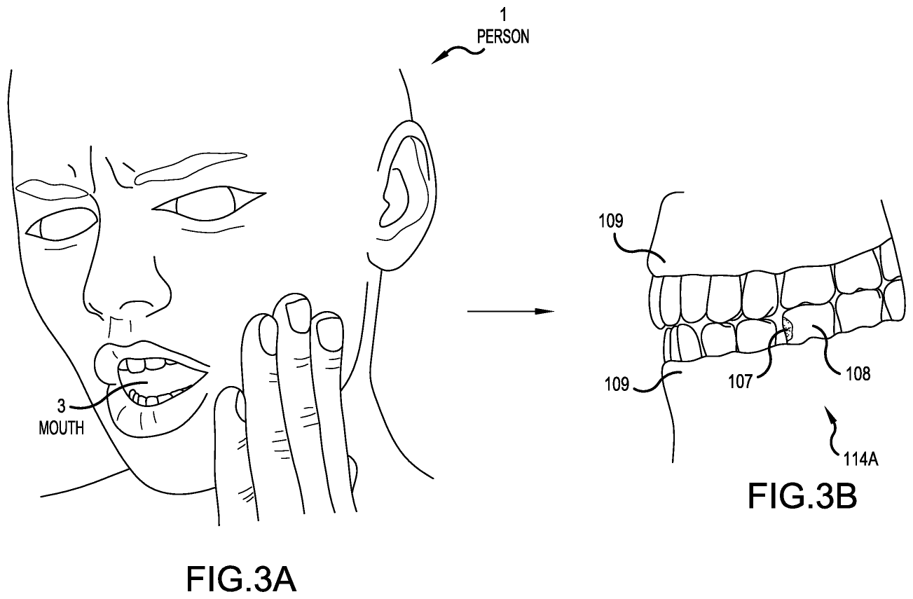

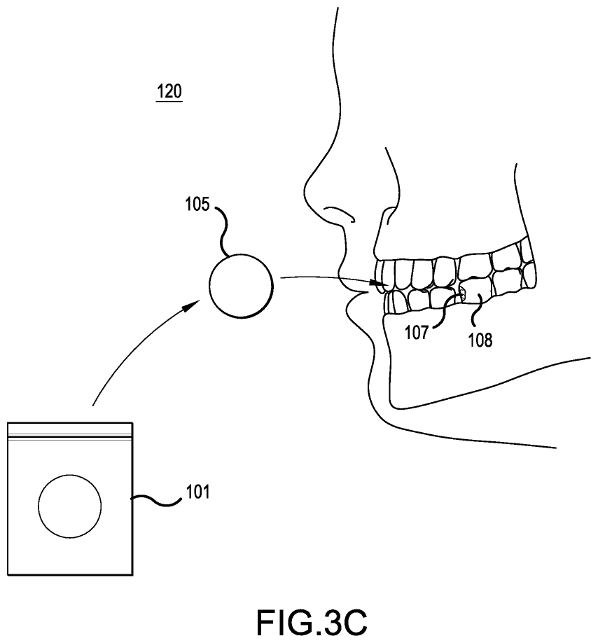

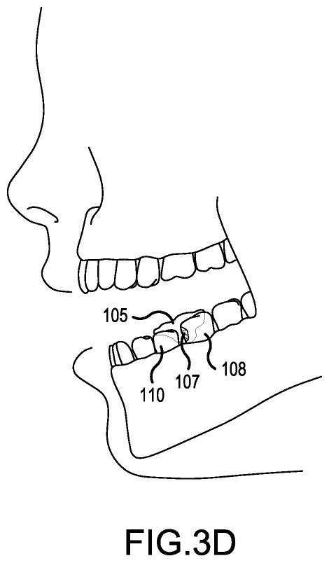

Referencing FIG. 3A, a person 1 is experiencing pain in their mouth 3. Referencing FIG. 3B the mouth 3 of the person 1 includes gums 109 and an affected tooth 108 with a fracture 107 that has caused a hole in the affected tooth 108.

Referencing FIG. 3C, a sequence 120 is shown in which a portion 102 of plug product is torn off from a block of plug product 101 and shaped into a ball (e.g. a sphere-like shape) to form shaped plug product 105 that is poised for application to the fracture 107 of the affected tooth 108. The fracture 107 of the affected tooth 108 is a source of tooth-associated pain

Referencing FIG. 3D the shaped plug product 105 has been applied to the fracture 107 to cover the hole in the affected tooth 108. As shown the applied plug product 105 covers portions of the affected tooth 108 and well as neighboring teeth 110

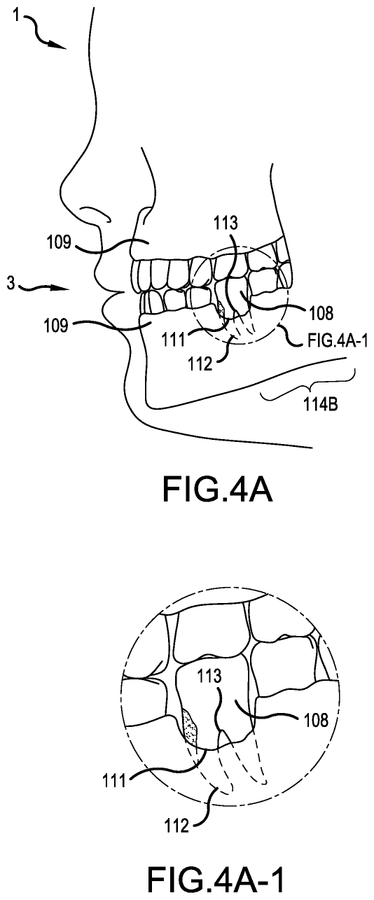

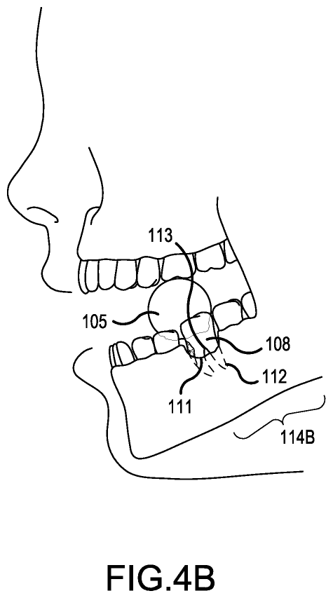

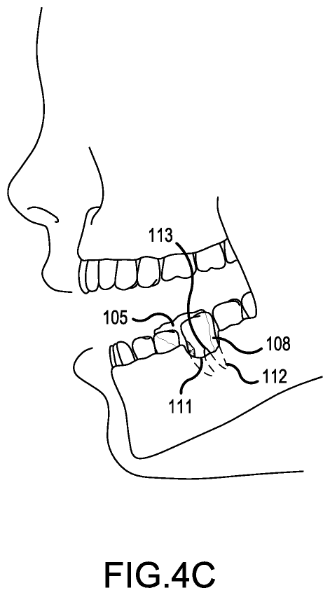

Referencing FIG. 4A, a mouth 3 of a person 1 includes an affected tooth 108 and gums 109. A source of tooth-associated pain 114B includes a receding gumline 111, a tooth root 112, and a partially exposed portion 113 of tooth root 112, all associated with tooth 108. The above structures are shown in an enlarged view in FIG. 4A-1. The location of the enlarged view of FIG. 4A-1 is shown in FIG. 4A.

Referencing FIG. 4B, a shaped plug product 105 is positioned to be applied directly to the source of tooth-associated pain 114B, including to the receding gumline 111 and to the partially exposed portion 113 of the tooth root 112, all associated with tooth 108.

Referencing FIG. 4C, a shaped plug product 105 has been applied directly to the source of tooth-associated pain 114B, including to the receding gumline 111 and to the partially exposed tooth root 112, both associated with affected tooth 108.

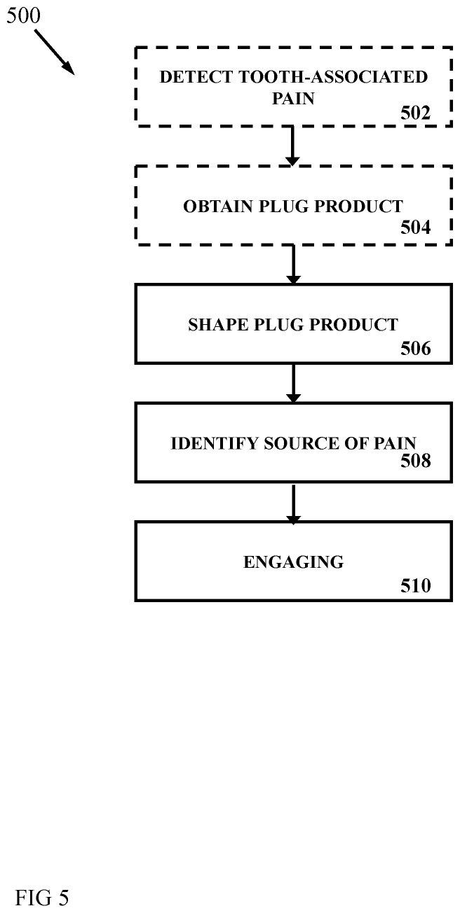

Referencing FIG. 5, in some embodiments a method 500 of relieving tooth-associated pain includes at least operation 506 of shaping plug product into a shape, operation 508 of identifying a source of tooth-associated pain, and operation 510 of engaging the shaped plug product directly with the source of tooth-associated pain.

Continuing with reference to FIG. 5, in some embodiments the method 500 optionally includes operation 502 of detecting the tooth-associated pain.

Continuing with reference to FIG. 5, in some embodiments the method 500 optionally includes operation 504 of obtaining the plug product. For example, in some embodiments a user obtains some plug product from storage, such as from a medicine cabinet or from a night stand drawer.

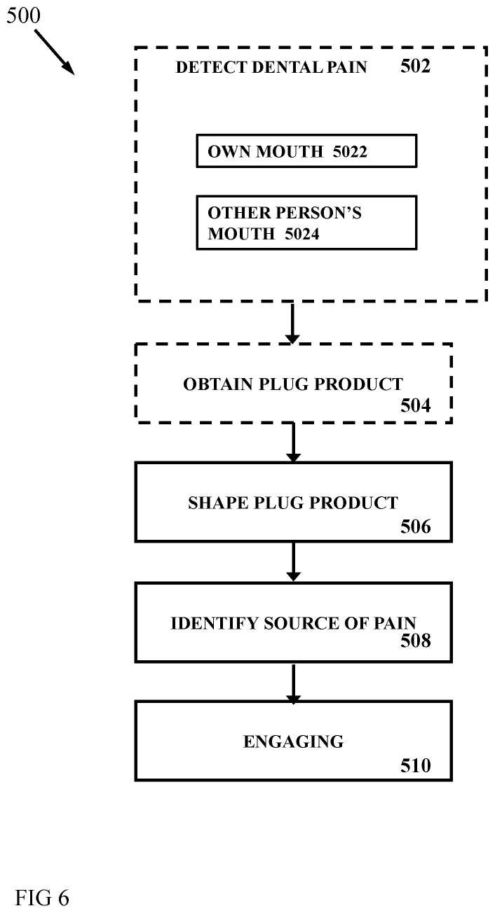

Referencing FIG. 6, in some embodiments, operation 502 includes operation 5022 of a person detecting tooth-associated pain by experiencing tooth-associated pain in their own mouth. Alternatively, in some embodiments operation 502 includes operation 5024 of a person receiving one or more communications about the tooth-associated pain from another person. For example, a person may be a friend of a person or a health care provider and the person receives one or more communications about the tooth-associated pain from another person, the other person being, for example, a child, a spouse, a patient, or other person.

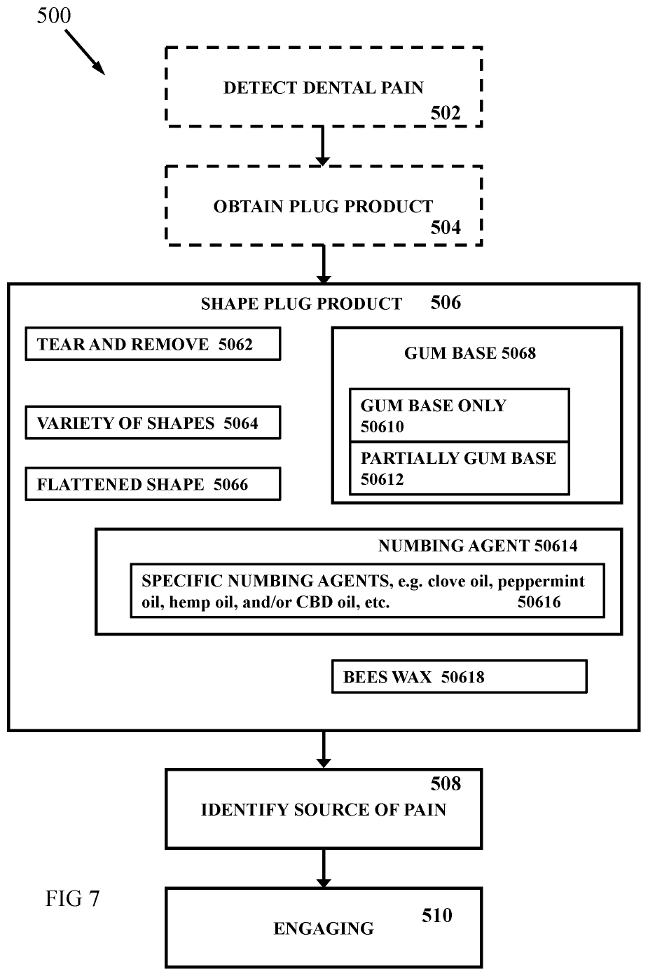

Referencing FIG. 7, in some embodiments operation 506 includes at least one of operations 5062, 5064, 5066, 5068, 50614, or 50618. Turning to operation 5062, in some embodiments shaping plug product into a shape includes at least tearing or removing an amount of plug product from a larger amount of plug product. The size of the torn-off or removed amount varies according to preference. But in some embodiments the removed or torn-off amount of plug product is approximately ½ inches in diameter. In some other embodiments the removed or torn-off amount is a cube with edges of approximately ½ inch in length.

Turning to operation 5064, in some embodiments shaping plug product into a shape includes at least shaping an amount of plug product into at least one of a sphere-like shape, ellipsoidal-like shape, or a rectangular-like shape. Turning to operation 5066, in some embodiments shaping plug product into a shape includes at least shaping an amount of plug product into a substantially flat shape. In some embodiments “substantially flat” refers to a shape having surfaces within 40% of true flat.

Turning to operation 5068, in some embodiments shaping plug product into a shape includes at least shaping a plug product that is at least partly composed of gum base. Gum base is insoluble and not nutritious. Gum base includes various compositions that may include one or more of food-grade polymers, plasticizers, softeners, texturizers, emulsifiers, or other ingredients. There are various commercial sources of gum base. For example, one product is “Ready Gum Base” sold by High Tech Gum Base Private Limited. Another example is “Gum base mixer chewing gum” or “bubble gum base” sold by Wuxi Gum Base Co. Ltd. Either of these products would be acceptable for some embodiments either with or without additional ingredients.

Turning to operation 50614, in some embodiments, shaping plug product into a shape includes at least in some embodiments shaping a plug product that is a mixture that includes at least (1) one or more numbing agents and (2) at least one of gum base or beeswax.

Turning to operation 50618, in some embodiments, shaping plug product into a shape includes at least shaping a plug product that is at least partly composed of beeswax.

Returning to discuss operation 5068, in some particular embodiments operation 5068 includes at least one of operation 50610 or 50612.

Turning to operation 50610, in some particular embodiments shaping a plug product that is at least partly composed of gum base includes at least shaping a plug product that is completely composed of gum base. Turning to operation 50612, in some particular embodiments shaping a plug product that is at least partly composed of gum base includes at least shaping a plug product that includes at least a mixture of at least (1) gum base and (2) at least one of stevia, corn starch, glycerin, citric acid, one or more numbing agents, corn syrup, or beeswax.

Returning to discuss operation 50614, in some particular embodiments operation 50614 includes at least operation 50616. Turning to operation 50616, in some embodiments shaping a plug product that is a mixture that includes at least (1) one or more numbing agents and (2) at least one of gum base or beeswax includes at least shaping a plug product wherein the one or more numbing agents include at least one of clove oil, peppermint oil, hemp oil, or cannabidiol (CBD) oil. For example, in some embodiments a numbing agent may be a synthetic product.

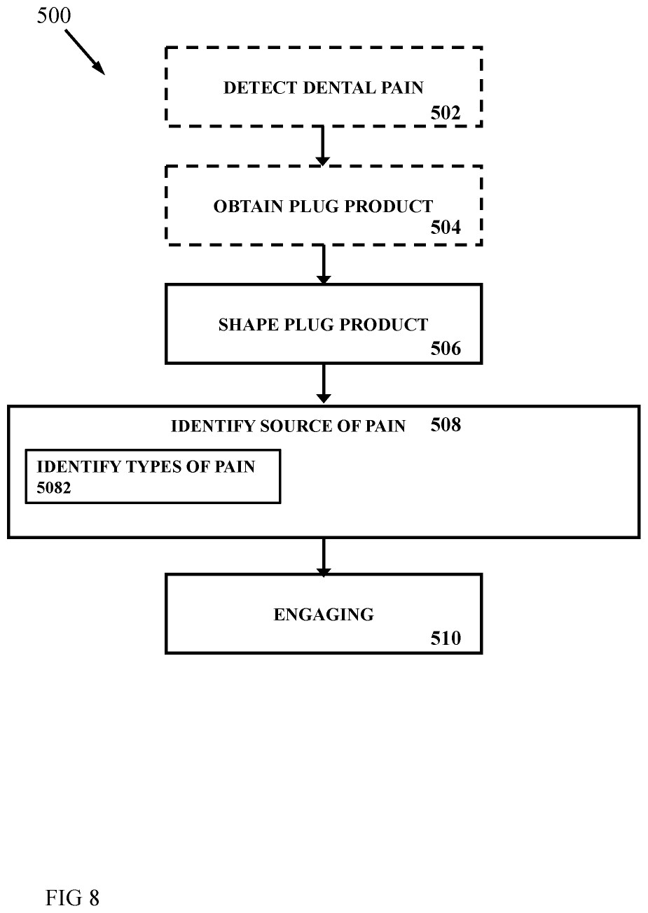

Referencing FIG. 8, in some embodiments operation 508 includes operation 5082. Turning to operation 5082, in some embodiments identifying a source of tooth-associated pain includes at least identifying at least one of a tooth cavity, a tooth with a missing filling, a tooth that is at least one of cracked or fractured, an at least partly exposed tooth root, or an exposed oral nerve. For example, a user may have a filling fall out leaving a hole through the enamel that potentially exposes a nerve to air, cold, or heat, causing nerve pain. Or, as a further example, tooth may fracture creating an hole that at least partially exposes a nerve to air, cold or warm temperatures, or other conditions. In a yet further example, a tooth root may become exposed due to a receding gum line.

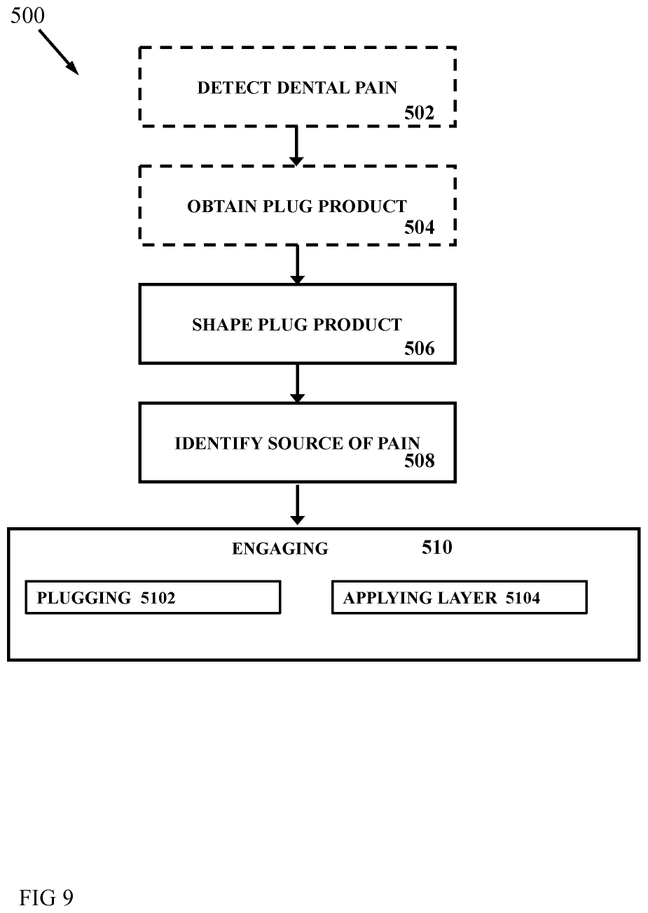

Referencing FIG. 9, in some embodiments operation 510 includes at least one of operation 5102 or operation 5104. Turning to operation 5102 in some embodiments engaging the shaped plug product directly with the source of tooth-associated pain includes at least with a shaped plug product, plugging a source of tooth-associated pain that is at least one of a tooth cavity, a tooth with a missing filling, a hole in a tooth, at least one of a tooth crack a tooth fracture, or an exposed oral nerve. For example, in some embodiments an amount of plug product is rolled into a ball and then a user uses there fingers to press the against to a source of tooth-associated pain, such as for example, against a crack on a tooth or a hole in a tooth caused by a missing filling or by a chip. The applied plug product then at least partially seals the crack or the hole to reduce exposure of any nerves to the oral environment, including any air or hot or cold temperatures. See, for example, plug product applied to a source of tooth-associated pain in FIGS. 3D and 4C.

Turning to operation 5104, in some embodiments engaging the shaped plug product directly with the source of tooth-associated pain includes at least applying a layer of shaped plug product directly to at least one of an exposed tooth root or an exposed oral nerve. For example, due to gum disease or other cause, a gum line associated with a tooth retracts, exposing at least some tooth root. Then exposed tooth root may cause aggravation of a dental nerve causing nerve pain. Therefore, in some embodiments an amount of plug product is shaped into a flat shape and then a user uses their fingers to apply the shaped plug product against the exposed tooth root. The applied plug product then at least partially seals the exposed tooth root to reduce exposure of any nerves to the oral environment, including any air or hot or cold temperatures.

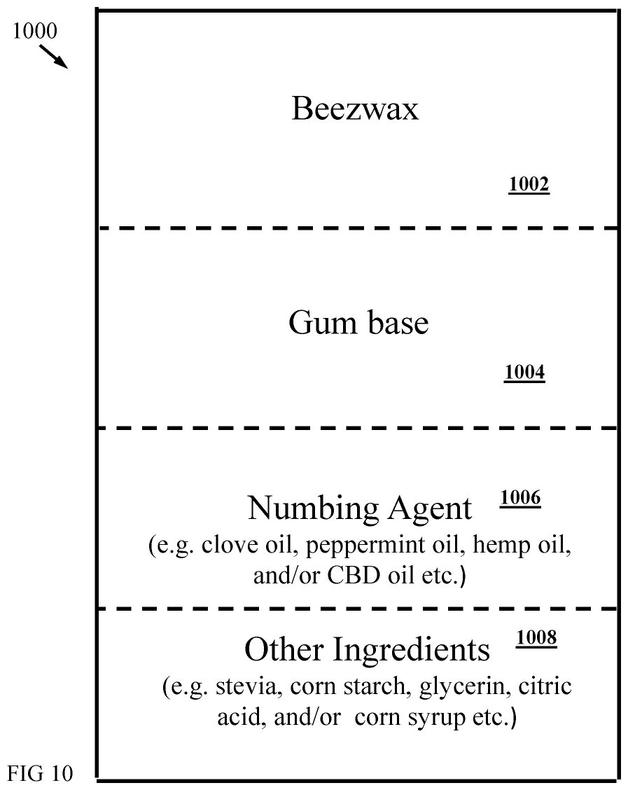

Referencing FIG. 10, in some embodiments a plug product 1000 is a mixture, that includes at least one or more numbing agents 1006, at least one of beeswax 1002 or gum base 1004, and other ingredients 1008 that at least one of stevia, corn starch, glycerin, citric acid, or corn syrup. Although FIG. 10 depicts elements 1002, 1004, 1006, and 1008 in some embodiments one or more of these elements are missing—they are not all required as various combinations are possible. Further, although the various elements 1002, 1004, 1006, 1008, are shown as separate blocks, this is merely for ease of presentation. In actual embodiments the elements are at least to some extent mixed together. In addition, FIG. 10 is not necessarily to scale. The size of the blocks defined by the lines is not intended to represent the amounts of the various ingredients.

Continuing with reference to FIG. 10, in some embodiments, the one or more numbing agents include at least one of clove oil, peppermint oil, hemp oil, or cannabidiol (CBD) oil.

Various embodiments are within the scope of this disclosure. For example in one embodiment a plug product is a mixture of 5 pounds of bees wax, two pounds of a gum base, a 16 ounce bottle of corn syrup and between 5 and 15 ounces of clove oil. The above measurements are approximate and can be varied according to preference. In some embodiments, other amounts of the various ingredients are used according to preference.

In one embodiment a mixture is prepared by melting 2 ounces of gum-base pellets and 1 tablespoon of corn syrup for each pound of beeswax (beeswax not mixed in yet) in a microwave in 10-second increments. The above mixture of gum base and corn syrup is then stirred after the ten second increments. The melted and stirred gum base and corn syrup is then mixed in with the beeswax. And this mixture is heated. When this mixture reaches 150 degrees Fahrenheit a desired amount of clove oil is added. The amount of clove oil may vary depending on preference but in some embodiments is between 5 and 15 ounces. The resulting mixture is then shaped to preference and cooled in a freezer or refrigerator for approximately 30 minutes.

In another embodiment without the one or more numbing agents and measured by volume instead of weight, the mixture is a ⅓ cup of gum base, a ¼ teaspoon of citric acid, 1 teaspoon vegetable glycerin, and one ¼ teaspoon bees wax. All of the above is mixed and rolled into corn starch.

Various embodiments are now discussed.

Embodiment 1. A method of reducing tooth-associated pain, the method comprising:
shaping plug product into a shape;
identifying a source of tooth-associated pain; and
engaging the shaped plug product directly with the source of tooth-associated pain.

Embodiment 2. The method of embodiment 1, further comprising:
obtaining the plug product.

Embodiment 3. The method of any of embodiments 1 or 2, further comprising:
detecting the tooth-associated pain.

Embodiment 4. The method of embodiment 3, wherein the detecting tooth-associated pain comprises:
a person experiencing tooth-associated pain in their own mouth.

Embodiment 5. The method of any of embodiments embodiment 3, wherein the detecting tooth-associated pain comprises:
a person receiving one or more communications about the tooth-associated pain from another person.

Embodiment 6. The method of any of embodiments 1— 5, wherein the shaping plug product into a shape comprises:
tearing or removing an amount of plug product from a larger amount of plug product.

Embodiment 7. The method of any of embodiments 1-6, wherein the shaping plug product into a shape comprises:
shaping an amount of plug product into at least one of a sphere-like shape, ellipsoidal-like shape, or a rectangular-like shape.

Embodiment 8. The method of any of embodiments 1-6, wherein the shaping plug product into a shape comprises:
shaping an amount of plug product into a substantially flat shape.

Embodiment 9. The method of any of embodiments 1-8, wherein the shaping plug product into a shape comprises:
shaping a plug product that is at least partly composed of gum base.

Embodiment 10. The method of embodiment 9, wherein the shaping a plug product that is at least partly composed of gum base comprises:
shaping a plug product that is completely composed of gum base.

Embodiment 11. The method of embodiment 9, wherein the shaping a plug product that is at least partly composed of gum base comprises:
shaping a plug product that includes at least a mixture of at least (1) gum base and (2) at least one of stevia, corn starch, glycerin, citric acid, one or more numbing agents, corn syrup, or beeswax.

Embodiment 12. The method of any of embodiments 1-8, wherein the shaping plug product into a shape comprises:
shaping a plug product that is at least partly composed of beeswax.

Embodiment 13. The method of any of embodiment 1-8, wherein the shaping plug product into a shape comprises:
shaping a plug product that is a mixture that includes at least (1) one or more numbing agents and (2) at least one of gum base or beeswax.

Embodiment 14. The method of embodiment 13, wherein shaping a plug product that is a mixture that includes at least (1) one or more numbing agents and (2) at least one of gum base or beeswax comprises:
shaping a plug product wherein the one or more numbing agents include at least one of clove oil, peppermint oil, hemp oil, or cannabidiol (CBD) oil.

Embodiment 15. The method of any of embodiments 1-14, wherein identifying a source of tooth-associated pain comprises:
identifying at least one of a tooth cavity, a tooth with a missing filling, a tooth that is at least one of cracked or fractured, an at least partly exposed tooth root, or an exposed oral nerve.

Embodiment 16. The method of any of embodiments 1— 15, wherein engaging the shaped plug product directly with the source of tooth-associated pain comprises:
with a shaped plug product, plugging a source of tooth-associated pain that is at least one of a tooth cavity, a tooth with a missing filling, a hole in a tooth, at least one of a tooth crack a tooth fracture, or an exposed oral nerve.

Embodiment 17. The method of any of embodiments 1-15, wherein engaging the shaped plug product directly with the source of tooth-associated pain comprises:
applying a layer of shaped plug product directly to at least one of an exposed tooth root or an exposed oral nerve.

Embodiment 18. A method of reducing tooth-associated pain, the method comprising:
detecting tooth-associated pain;
shaping plug product into a shape;
identifying a source of tooth-associated pain; and
engaging the shaped plug product directly with the source of tooth-associated pain.

Embodiment 19. A plug product for reducing tooth-associated pain, the plug product comprising:
a mixture that includes at least:
one or more numbing agents;
at least one of beeswax or gum base; and
at least one of stevia, corn starch, glycerin, citric acid, or corn syrup.

Embodiment 20. The plug product of embodiment 19, wherein the one or more numbing agents include at least one of clove oil, peppermint oil, hemp oil, or cannabidiol (CBD) oil.

In some embodiments, a ratio of 70 percent beeswax and 30% gum base (by weight) is used. In some further embodiments, a raw yellow settled bees wax is used. In some embodiments the mixing of beeswax and gum base is aided by heat, for example from a heated room, from a microwave as discussed above, or from steam.

It will be understood by those skilled in the art that the terminology used in this specification and in the claims is “open” in the sense that the terminology is open to additional elements not enumerated. For example, the word “includes” should be interpreted to mean “including at least” and so on. Even if “includes at least” is used sometimes and “includes” is used other times, the meaning is the same: includes at least. In addition, articles such as “a” or “the” should be interpreted as not referring to a specific number, such as one, unless explicitly indicated. At times a convention of “at least one of A, B, or C” is used, the intent is that this language includes any combination of A, B, C, including, without limitation, any of A alone, B alone, C alone, A and B, B and C, A and C, all of A, B, and C or any combination of the foregoing, such as for example AABBC, or ABBBCC. The same is indicated by the conventions “one of more of A, B, or C” and “and/or”.

Although embodiments have been described in detail, it should be understood that various changes, substitutions, and alterations could be made hereto without departing from the spirit and scope of the invention as defined by the appended claims and equivalents thereof.

I claim:

1. A method of reducing tooth-associated pain, the method comprising:
shaping plug product into a shape;
identifying a source of tooth-associated pain; and
engaging the shaped plug product directly with the source of tooth-associated pain.

2. The method of claim 1, further comprising:
obtaining the plug product.

3. The method of claim 1, further comprising:
detecting the tooth-associated pain.

4. The method of claim 3, wherein the detecting tooth-associated pain comprises:
a person experiencing tooth-associated pain in their own mouth.

5. The method of claim 3, wherein the detecting tooth-associated pain comprises:
a person receiving one or more communications about the tooth-associated pain from another person.

6. The method of claim 1, wherein the shaping plug product into a shape comprises:
tearing or removing an amount of plug product from a larger amount of plug product.

7. The method of claim 1, wherein the shaping plug product into a shape comprises:
shaping an amount of plug product into at least one of a sphere-like shape, ellipsoidal-like shape, or a rectangular-like shape.

8. The method of claim 1, wherein the shaping plug product into a shape comprises:
shaping an amount of plug product into a substantially flat shape.

9. The method of claim 1, wherein the shaping plug product into a shape comprises:
shaping a plug product that is at least partly composed of gum base.

10. The method of claim 9, wherein the shaping a plug product that is at least partly composed of gum base comprises:
shaping a plug product that is completely composed of gum base.

11. The method of claim 9, wherein the shaping a plug product that is at least partly composed of gum base comprises:
shaping a plug product that includes at least a mixture of at least (1) gum base and (2) at least one of stevia, corn starch, glycerin, citric acid, one or more numbing agents, corn syrup, or beeswax.

12. The method of claim 1, wherein the shaping plug product into a shape comprises:
shaping a plug product that is at least partly composed of beeswax.

13. The method of claim 1, wherein the shaping plug product into a shape comprises:
shaping a plug product that is a mixture that includes at least (1) one or more numbing agents and (2) at least one of gum base or beeswax.

14. The method of claim 13, wherein shaping a plug product that is a mixture that includes at least (1) one or more numbing agents and (2) at least one of gum base or beeswax comprises:
shaping a plug product wherein the one or more numbing agents include at least one of clove oil, peppermint oil, hemp oil, or cannabidiol (CBD) oil.

15. The method of claim 1, wherein identifying a source of tooth-associated pain comprises:
identifying at least one of a tooth cavity, a tooth with a missing filling, a tooth that is at least one of cracked or fractured, an at least partly exposed tooth root, or an exposed oral nerve.

16. The method of claim 1, wherein engaging the shaped plug product directly with the source of tooth-associated pain comprises:
with a shaped plug product, plugging a source of tooth-associated pain that is at least one of a tooth cavity, a tooth with a missing filling, a hole in a tooth, at least one of a tooth crack a tooth fracture, or an exposed oral nerve.

17. The method of claim 1, wherein engaging the shaped plug product directly with the source of tooth-associated pain comprises:
applying a layer of shaped plug product directly to at least one of an exposed tooth root or an exposed oral nerve.

18. A method of reducing tooth-associated pain, the method comprising:
detecting tooth-associated pain;
shaping plug product into a shape;
identifying a source of tooth-associated pain; and
engaging the shaped plug product directly with the source of tooth-associated pain.

19. A plug product for reducing tooth-associated pain, the plug product comprising:
a mixture that includes at least:
one or more numbing agents;
at least one of beeswax or gum base; and
at least one of stevia, corn starch, glycerin, citric acid, or corn syrup.

20. The plug product of claim 19, wherein the one or more numbing agents include at least one of clove oil, peppermint oil, hemp oil, or cannabidiol (CBD) oil.
